(19) United States
(12) Patent Application Publication
(10) Pub. No.: US 2025/0195568 A1
(43) Pub. Date: Jun. 19, 2025

(54) BIOPOLYMER FORMULATIONS

(71) Applicant: Pandorum Technologies Private Limited, Bangalore (IN)

(72) Inventors: Tuhin BHOWMICK, New York, NY (US); Parinita AGRAWAL, Bangalore (IN); Suvro Kanti CHOWDHURRY, Bangalore (IN); Kamalnath SELVAKUMUAR, Bangalore (IN)

(21) Appl. No.: 18/929,049

(22) Filed: Oct. 28, 2024

Related U.S. Application Data
(63) Continuation of application No. PCT/IN2023/050417, filed on Apr. 28, 2023.

(30) Foreign Application Priority Data
Apr. 28, 2022 (IN) 202241025040

(51) Int. Cl. A61K 35/12; A61K 9/06; A61K 47/36; A61K 47/42; A61P 27/02; C08L 5/08; C08L 89/06

(52) U.S. Cl. CPC A61K 35/12; A61K 9/06; A61K 47/36; A61K 47/42; A61P 27/02; C08L 5/08; C08L 89/06

(57) ABSTRACT

Provided in the present disclosure are biopolymer formulations comprising thiolated gelatin and methacrylated hyaluronic acid. Said formulations are useful for corneal applications, by way of example for treatment of disease of or damage to a cornea in a subject. Also provided are methods for preparing the biopolymer formulations, and applications thereof.

### CROSS-REFERENCE TO RELATED APPLICATIONS

This application is a continuation of International Patent Application No. PCT/IN2023/050417, filed Apr. 28, 2023, which claims priority to Indian Patent Application No. 202241025040, filed Apr. 28, 2022 the entire contents of which are incorporated herein by reference.

### BACKGROUND

Corneal blindness is the fourth leading cause of blindness in the world and an estimated 1.5 million new cases have been reported worldwide each year. About 10 million people in the world are affected by bilateral corneal blindness and another 23 million with unilateral corneal blindness. The leading causes of corneal dysfunction include trachoma (involving scarring and vascularization of the cornea), ocular trauma, corneal ulceration, and infections, such as those due to herpes simplex virus. One of the key medical treatments for corneal diseases include keratoplasty (corneal transplant). However, there are various complications associated with cornea transplant, which includes: (i) keratoplasty patients experiences tissue (cornea) rejection; (ii) scarring from infections, such as eye herpes or fungal keratitis; (iii) glaucoma (increased pressure inside the eye); (iv) visual acuity problems (sharpness of the vision) caused by an irregular curve in the shape of the cornea; (v) detachment of the corneal transplant; (vi) high cost and inconveniences surrounding the safe extraction, storage, and transportation of living tissue.

Seeing the limitations associated with cornea transplant, various efforts have been made to minimize or eliminate the need to rely on keratoplasty, for example biopolymer-based ocular adhesives that, once applied to diseased, degenerated, damaged, or partially re-sectioned cornea, mimic the properties of natural cornea. Such ocular adhesives may be used by way of example for closure of ocular wounds after an injury or during corneal surgeries. In corneal surgeries, biopolymers may be employed as suture-less substitutes for closing perforations post-surgery. Various biomaterials are reported in the literature for treating corneal damage arising from eye diseases.

However, conventional ocular adhesives still suffer from suboptimal properties including, rapid polymerization and heat generation, low biocompatibility, low transparency and rough surfaces, difficulty in handling, short residence times and poor integration with host ocular tissues.

Thus, there remains a long-felt need in the art to develop an efficient, biocompatible, and biodegradable cross-linked hydrogel formulations to match characteristics of native cornea that would help in treating corneal diseases while avoiding side effects. Given the (1) remarkable physical characteristics of the cornea that combines a high degree of transparency and durability, and (2) the hard-to-predict effect on polymer chemistry and combinations on the physical properties of resulting hydrogels, generating a hydrogel that is compatible with, and matches the physical characteristics of, native cornea has remained a difficult challenge.

### SUMMARY

Provided herein are embodiments of a biopolymer formulation comprising a thiolated gelatin and a methacrylated hyaluronic acid.

In some variations, the thiolated gelatin may have an average molecular weight of between about 50 kDa and about 200 kDa, and an average degree of substitution of between about 0.2 mmol/g and about 1.2 mmol/g; and the methacrylated hyaluronic acid may have an average molecular weight of between 20 kDa and 80 kDa, and an average degree of substitution of between 0.5 mmol/g and 1 mmol/g.

In some variations, the biopolymer formulation may comprise: a thiolated gelatin having a bloom value of between about 200 bloom and about 275 bloom, and an average degree of substitution of between about 0.5 mmol/g and about 0.9 mmol/g; and a methacrylated hyaluronic acid having an average molecular weight of between 25 kDa and 40 kDa, and an average degree of substitution of between about 0.7 mmol/g and about 0.9 mmol/g, wherein the thiolated gelatin and the methacrylated hyaluronic acid are at a weight ratio of between about 100:30 and about 100:70. Optionally, the thiolated gelatin may have a bloom value of between about 220 bloom and about 230 bloom and an average degree of substitution between about 0.5 mmol/g and about 0.7 mmol/g, and the thiolated gelatin and the methacrylated hyaluronic acid may be at a weight ratio of between about 100:55 and about 100:65. Optionally, the thiolated gelatin may have a bloom value of between about 240 bloom and about 260 bloom and an average degree of substitution between about 0.7 mmol/g and about 0.9 mmol/g, and the thiolated gelatin and the methacrylated hyaluronic acid may be at a weight ratio of between about 100:35 and about 100:45.

In some variations, the biopolymer formulation may be in a form of a dry pre-mix, wherein the methacrylated hyaluronic acid and the thiolated gelatin are in dry powder form.

In some variations, the biopolymer formulation may be in the form of a liquid or a hydrogel and may comprise a saline solution and at least one photoinitiator compound.

There is also provided herein embodiments of method of treating a disease of or damage to a cornea of a subject. In some variations, method may comprise: mixing a dry biopolymer pre-mix comprising a thiolated gelatin and a methacrylated hyaluronic acid with a photoinitiator solution to create a liquid biopolymer mixture; applying the liquid biopolymer mixture to the cornea of the subject; and exposing the liquid biopolymer mixture applied on the cornea to light having a wavelength within an excitation range of the at least one photoinitiator. In some variations, the dry biopolymer pre-mix may comprise: a thiolated gelatin optionally having an average molecular weight between 50 kDa and 200 kDa, and an average degree of substitution of between 0.2 mmol/g and 1.2 mmol/g; and a methacrylated hyaluronic acid optionally having an average molecular weight between 20 kDa and 80 kDa, and an average degree of substitution of between 0.5 mmol/g and 1 mmol/g.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations.

FIGS. 1A-1C show hCSSCs grown in coverslip cultures at 1 day (FIG. 1A), 2 weeks (FIG. 1B) and 4 weeks (FIG. 1C) after culturing and stained for Calcein-AM, Ethidium homodimer, and DAPI to evaluate cell viability.

FIGS. 1D-1F show hCSSCs grown within a gel-SH hydrogel at 1 day (FIG. 1D), 2 weeks (FIG. 1E) and 4 weeks (FIG. 1F) after culturing and stained for Calcein-AM, Ethidium homodimer, and DAPI to evaluate cell viability.

FIGS. 2A-2C show hCSSCs grown in coverslip cultures at 1 day (FIG. 2A), 2 weeks (FIG. 2B) and 4 weeks (FIG. 2C) after culturing and stained for CD90, αSMA, DAPI to evaluate cell differentiation.

FIGS. 2D-2F show hCSSCs grown within a gel-SH hydrogel at 1 day (FIG. 2D), 2 weeks (FIG. 2E) and 4 weeks (FIG. 2F) after culturing and stained for CD90, αSMA, DAPI to evaluate cell differentiation.

FIGS. 3A-3B show phase contrast images of hCEC cells grown in the coverslip cultures after 4 days comparing hCECs grown on a coverslip surface (FIG. 3A) and on a hydrogel surface (FIG. 3B).

FIGS. 4A-4C show hCEC cells grown on a coverslip surface and stained with DAPI (FIG. 4A), ZO1 (FIG. 4B) and rhodamine-phalloidin (FIG. 4C).

FIGS. 4D-4F show hCEC cells grown on a hydrogel surface and stained with DAPI (FIG. 4D), ZO1 (FIG. 4E) and rhodamine-phalloidin (FIG. 4F).

### DETAILED DESCRIPTION

Non-limiting examples of various aspects and variations of the invention are described herein.

There is provided herein embodiments of a biopolymer formulation. The biopolymer formulation may, in some variations, be used to produce a hydrogel that is compatible with, and matches physical characteristics of, native cornea.

In some variations, the biopolymer formulation comprises a thiolated gelatin (gel-SH) and a methacrylated hyaluronic acid (HA-MA). Gel-SH is a gelatin that is modified with a thiol group. HA-MA is a hyaluronic acid modified with a methacrylate. Without being bound to theory or mechanism, modification of a polymer such as hyaluronic acid with a methacrylate allows the polymer to become crosslinked. The crosslinking may be a di-methacrylate crosslinking between two methacrylate groups (by way of example between two HA-MA molecules), a thiol-ene crosslinking between a methacrylate group and a thiol group (by way of example between a gel-SH molecule and an HA-MA molecule), or a combination thereof. The crosslinking may be induced through a photoinitiator compound in the presence of light. Examples of photoinitiator compounds for use in methacrylate crosslinking include an eosin, by way of example eosin Y, and/or triethanolamine.

### Gel-SH Properties

In some variations, the gel-SH of a biopolymer formulation of the disclosure may have an average molecular weight of between about 50 kDa and about 200 kDa, between about 50 kDa and about 100 kDa, between about 40 kDa and about 120 kDa, between about 60 kDa and about 90 kDa, between about 60 kDa and about 150 kDa, about 50 kDa, about 60 kDa, about 70 kDa, about 80 kDa, about 90 kDa, about 100 kDa, about 120 kDa, about 140 kDa, about 160 kDa, about 180 kDa, and about 200 kDa. The average molecular weight of the gel-SH may be determined by size exclusion chromatography (SEC), or based on the bloom value that may be determined with a gelometer.

In some variations, the gel-SH comprised in the formulation may have an average degree of substitution of between about 0.2 mmol/g and about 1.2 mmol/g, between about 0.2 mmol/g and about 0.9 mmol/g, between about 0.3 mmol/g and about 0.9 mmol/g, between about 0.5 mmol/g and about 0.9 mmol/g, between about 0.3 mmol/g and about 0.8 mmol/g, between about 0.3 mmol/g and about 0.7 mmol/g, between about 0.65 mmol/g and about 0.75 mmol/g, about 0.2 mmol/g, about 0.3 mmol/g, about 0.3 mmol/g, about 0.4 mmol/g, about 0.5 mmol/g, about 0.6 mmol/g, about 0.62 mmol/g, about 0.64 mmol/g, about 0.66 mmol/g, about 0.68 mmol/g, about 0.7 mmol/g, about 0.72 mmol/g, about 0.75 mmol/g, about 0.8 mmol/g, about 0.9 mmol/g, about 1 mmol/g, about 1.1 mmol/g, or about 1.2 mmol/g.

In some variations, the gel-SH has a bloom value of between about 100 bloom and about 300 bloom, between about 200 bloom and about 300 bloom, between about 100 bloom and about 150 bloom, between about 150 bloom and about 250 bloom, between about 200 bloom and about 250 bloom, between about 200 bloom and about 275; between about 220 bloom and about 260 bloom, between about 220 bloom and about 280 bloom, between about 210 bloom and about 240 bloom, about 100 bloom, about 120 bloom, about 125 bloom, about 150 bloom, about 175 bloom, about 200 bloom, about 215 bloom, about 225 bloom, about 240 bloom, about 250 bloom, about 260 bloom, about 270 bloom, about 280 bloom, about 290 bloom, or about 300 bloom.

### HA-MA Properties

In some variations, the HA-MA of a biopolymer formulation of the disclosure may have an average molecular weight of between about 20 kDa and about 80 kDa, between about 30 kDa and about 60 kDa, between about 20 kDa and about 50 kDa, between about 30 kDa and about 40 kDa, between about 30 kDa and about 35 kDa, about 25 kDa, about 28 kDa, about 30 kDa, about 33 kDa, about 35 kDa, about 40 kDa, about 45 kDa, about 50 kDa, about 60 kDa, about 70 kDa, and about 80 kDa. The average molecular weight of the HA-MA may be determined by size exclusion chromatography (SEC).

In some variations, the HA-MA of a biopolymer formulation of the disclosure may have an average degree of substitution of between about 0.5 mmol/g and about 1 mmol/g, between about 0.6 mmol/g and about 1 mmol/g, between about 0.7 mmol/g and about 0.9 mmol/g, about 0.5 mmol/g, about 0.6 mmol/g, about 0.7 mmol/g, about 0.75 about 0.8 mmol/g, 0.81 mmol/g, about 0.85 mmol/g, and about 0.9 mmol/g.

### Dry Pre-Mix

In some variations, the biopolymer formulations of the disclosure may be in the form of a dry pre-mix preparation, wherein the gel-SH and the HA-MA are in a dry powder form, optionally lyophilized. In some variations, the gel-SH and the HA-MA may be evenly mixed and stored in a sterile container ready for reconstitution with water, a physiologically compatible saline solution, optionally a buffered saline; such reconstitution can take place at the site of the corneal procedure, e.g. at the bedside. Optionally, the gel-SH and the HA-MA are pre-mixed in solution form. The gel-SH and the HA-MA in a desired ratio may undergo a preliminary solubilization together in water or a saline, then dried (lyophilized) after solubilization so that the resulting powder is a thoroughly mixed mixture of gel-SH and HA-MA. The preliminary solubilization may be conducted in a dark environment or in a container with light shielding, and/or without photo-initiators, in order to prevent premature cross-linking, so that the gel-SH and the HA-MA in the pre-mix remains in an un-crosslinked state after the preliminary solubilization and drying.

In some variations, the gel-SH and the HA-MA may be mixed in the dry pre-mix at a weight ratio (gel-SH:HA-MA) of between about 100:30 and about 100:90, between about 100:30 and about 100:70, between about 100:30 and about 100:60, between about 100:40 and about 100:70; between about 100:50 and about 100:70, about 100:40, about 100:50, about 100:60, about 100:70, or about 100:80.

In some variations, the pre-mix may additionally comprise a therapeutic agent, also in dried or lyophilized form. Exemplary therapeutic agents include, but are not limited to, exosomes and liposomes.

### Hydrogel

In some variations, the biopolymer formulations of the disclosure may be in the form of a liquid biopolymer mixture in which the gel-SH and HA-MA are not (or minimally) crosslinked or a hydrogel in which the gel-SH and the HA-MA are generally crosslinked, with the gel-SH and the HA-MA in an aqueous solution. In the liquid biopolymer mixture or hydrogel form, the biopolymer formulation may comprise an aqueous solvent. In some variations, the aqueous solvent may be water or saline. The saline may be a physiological saline having an osmolality or osmolarity compatible with a tissue, e.g. a cornea. The saline may be a buffered saline, such as a phosphate buffered saline (PBS).

In some variations, the gel-SH and HA-MA may be crosslinked to form the hydrogel. The crosslinks may comprise di-methacrylate crosslinks between HA-MA molecules and/or thiol-ene crosslinks between a gel-SH molecule and an HA-MA molecule. The HA-MA in the liquid biopolymer liquid or hydrogel may be between about 0% and about 100% crosslinked, between about 10% and about 90% crosslinked, between about 20% and about 80% crosslinked, not cross-linked, about 5% crosslinked, about 10% crosslinked, about 15% crosslinked, about 20% crosslinked, about 25% crosslinked, about 30% crosslinked, about 40% crosslinked, about 50% crosslinked, about 60% crosslinked, about 70% crosslinked, about 80% crosslinked, about 90% crosslinked, or about 100% crosslinked.

In some variations, a biopolymer formulation in liquid biopolymer mixture or hydrogel form may comprise a gel-SH at a concentration of between about 50 mg/ml and about 140 mg/ml, between about 70 mg/ml and about 140 mg/ml, between about 75 mg/ml and about 125 mg/ml, between about 90 mg/ml and about 110 mg/ml, between about 90 mg/ml and about 130 mg/ml, between about 100 mg/ml and about 120 mg/ml, about 70 mg/ml, about 75 mg/ml, about 80 mg/ml, about 90 mg/ml, about 100 mg/ml, about 110 mg/ml, about 120 mg/ml, about 125 mg/ml, about 130 mg/ml, or about 140 mg/ml.

In some variations, a biopolymer formulation in liquid biopolymer mixture or hydrogel form may comprise a HA-MA at a concentration of between about 40 mg/ml and about 90 mg/ml, about 40 mg/ml and about 70 mg/ml, between about 45 mg/ml and about 65 mg/ml, between about 50 mg/ml and about 60 mg/ml, about 40 mg/ml, about 45 mg/ml, about 50 mg/ml, about 55 mg/ml, about 60 mg/ml, about 65 mg/ml, about 70 mg/ml, about 75 mg/ml, or about 80 mg/ml.

In some variations, a biopolymer formulation in liquid biopolymer mixture or hydrogel form may comprise at least one photoinitiator. Photoinitiators are generally a compound or a combination of compounds that catalyze cross-linking of functionalized polymers such as HA-MA and/or gel-SH. In some variations, the at least one photoinitiator comprises an eosin, triethanolamine, or a combination thereof. In some variations, the eosin is an eosin Y. In some variations, the biopolymer formulation may comprise eosin Y at a concentration of between about 0.02 mM and about 0.2 mM, between about 0.05 mM and about 0.2 mM, between about 0.05 mM and about 0.1 mM, about 0.05 mM, about 0.08 mM, about 0.1 mM, or about 1.5 mM. In some variations, the biopolymer formulation may comprise eosin Y at a concentration of between about 0.02% v/v and about 0.2% v/v, between about 0.05% v/v and about 0.2% v/v, between about 0.02% v/v and about 0.1% v/v, between about 0.05% v/v and about 0.1% v/v, between about 0.07% v/v and about 0.09% v/v, about 0.6% v/v, about 0.7% v/v, about 0.76% v/v, about 0.8% v/v, about 0.9% v/v or about 1% v/v.

In some variations, the biopolymer formulation in liquid biopolymer mixture or hydrogel form may comprise a therapeutic agent. The therapeutic agent may be a therapeutic exosome or liposome.

### Methods of Producing Biopolymer Hydrogel

In some variations, a biopolymer hydrogel in accordance with embodiments of the disclosure may be produced by mixing a dry biopolymer pre-mix and a photoinitiator solution to create a liquid biopolymer mixture, applying the liquid biopolymer mixture to a surface, and exposing the liquid biopolymer mixture applied on the surface to light having a wavelength within an excitation (absorption) range of the photoinitiator. By way of example, the excitation range of an exemplary photoinitiator, eosin Y, is between about 440 nm and about 575 nm, with a peak absorption of about 525 nm. The light may be a white light comprising a mix of wavelengths, including wavelengths within the excitation range of the photoinitiator.

In some variations, the liquid biopolymer mixture may comprise a therapeutic agent that is mixed together with the dry biopolymer pre-mix and the photoinitiator solution. The therapeutic agent may be mixed in dried or lyophilized form optionally comprised in the pre-mix, comprised in a second solution, or comprised in the photoinitiator solution. In some variations, the therapeutic agent may be a therapeutic exosome or liposome.

The surface upon which the liquid biopolymer mixture is applied, then crosslinked with light exposure to form the crosslinked hydrogel, may be a biological surface. The biological surface may be a tissue, by way of example a cornea. The cornea may be a diseased, degenerated, damaged, or partially re-sectioned cornea.

### Methods of Treatment

Biopolymer formulations of the disclosure in the form of a hydrogel exhibit one or more of the following improvements over conventional hydrogels that are used as ocular adhesives: improved transparency, improved adhesive strength, lower swelling (as measured by weight and/or volume), reduced degradation over time, compressive modulus more compatible with tissue such as cornea, and improved biocompatibility with cells such as corneal cells. As such, it may be advantageous to use biopolymer formulations of the disclosure as an ocular adhesive or temporary corneal replacement to treat damage to the cornea or a corneal disease.

In some variations, a method of treating a disease of or damage to a cornea of a subject may comprise: providing a liquid biopolymer mixture of the disclosure comprising a photoinitiator; applying the liquid biopolymer mixture to a corneal wound site of a subject; and exposing the liquid biopolymer mixture applied on the cornea to light having a wavelength within an excitation range of the photoinitiator, so that the liquid biopolymer mixture is crosslinked and formed within the corneal wound site into a hydrogel replacement cornea (HRC). The corneal wound site may be a wound site surgically excavated by, for example, corneal trephination, to remove a diseased, scarred, or damaged corneal tissue. The HRC may provide an environment that stimulates corneal regeneration, such that the HRC is replaced over time by a regenerated corneal tissue formed at the corneal wound site.

In some variations, the liquid biopolymer mixture may be applied to a subject's eye as follows: (1) the diseased, damaged, or scarred cornea is surgically excavated and removed, by way example by corneal trephination; (2) an appropriate volume of the liquid biopolymer mixture is applied to fill the gap in corneal tissue; (3) white light of appropriate intensity is projected on to the application site to crosslink the liquid biopolymer mixture and convert the mixture into the HRC. In some variations, after the HRC is formed, the site may be washed with saline and treated with an antibiotic, by way of example moxifloxacin. In some variations, the cornea may be covered with a bandage contact lens for additional protection of the surgical site.

In some variations, the white light to crosslink the liquid biopolymer mixture may be applied at an intensity of between 1 mW/cm² and 100 mW/cm², between 10 mW/cm² and 100 mW/cm², between 5 mW/cm² and 50 mW/cm², between 5 mW/cm² and 20 mW/cm², between 8 mW/cm² and 12 mW/cm², between 80 mW/cm² and 100 mW/cm², about 5 mW/cm², about 8 mW/cm², about 10 mW/cm², about 12 mW/cm², about 15 mW/cm², about 20 mW/cm², about 30 mW/cm², about 40 mW/cm², about 60 mW/cm², or about 80 mW/cm², or about 100 mW/cm². The duration of the light application may be between 5 minutes (min) and 20 min, between 5 min and 10 min, between 6 min and 8 min, about 5 min, about 6 min, about 8 min, about 10 min, and about 12 min. A lower intensity may require a longer duration, and a shorter duration may be sufficient for a higher intensity.

In some variations, the liquid biopolymer mixture may be generated by mixing a dry biopolymer pre-mix of the disclosure and a photoinitiator solution of the disclosure.

In some variations, the liquid biopolymer mixture may comprise one or more therapeutic agents appropriate for treating the disease of or damage to the cornea. Optionally, the one or more therapeutic agents may comprise therapeutic exosome or liposome.

The corneal disease may be an anterior corneal scarring involving epithelial and stromal injuries/infection (active inflammation), a Stage 1 neurotrophic keratitis (NK) (persistent corneal epithelial defect), a Stage 2 NK (large persistent epithelial defect characterized by smooth, rolled edges), a Stage 3 NK (deep corneal ulcer, stromal melting, and sterile hypopyon), a corneal ulcer such as Mooren's ulcer, keratoconus, a corneal perforation, or corneal damage due to dry eye. The corneal disease may include corneal a limbal injuries and corneal dystrophy (CD), such as a lattice CD type 1, a granular CD type 1, and a congenital stromal CD, wherein the corneal stroma is damaged in the subject. The corneal disease may include a Schnyder CD or a lattice CD type 2, wherein both the epithelium and stroma are compromised.

### EXAMPLES

### Example 1—Production of Liquid Biopolymer Formulation and Hydrogel

A first exemplary liquid biopolymer formulation (“Formula 1”) was produced as follows: A dry pre-mix comprising a gel-SH (research grade bovine gelatin from Sigma-Aldrich; functionalized by Blafar Ltd.) and an HA-MA (HA from Stanford Chemicals; functionalized by Blafar Ltd.) at a 100:60 weight ratio (gel-SH:HA-MA) was produced. The gel-SH had an average molecular weight of between 50 kDa and 100 kDa (about 225 bloom) and an average degree of substitution of about 0.6 mmol/g. The HA-MA had an average molecular weight of about 33 kDa and an average degree of substitution of about 0.81 mmol/g. The gel-SH/HA-MA dry pre-mix was combined with a photoinitiator solution comprising eosin Y and triethanolamine in a saline solution. The resulting liquid biopolymer mixture comprised eosin Y at a concentration of 0.05-0.1 mM Eosin Y, triethanolamine at a concentration of about 0.076% w/v, gel-SH at a concentration of about 100 mg/ml and HA-MA at a concentration of about 60 mg/ml.

A second exemplary liquid biopolymer formulation (“Formula 2”) was produced as follows: A dry pre-mix comprising a gel-SH (clinical grade gelatin from beMatrix, Nitta; functionalized by Blafar Ltd.) and an HA-MA (HA from Stanford Chemicals; functionalized by Blafar Ltd.) at a 120:50 gel-SH:HA-MA weight ratio (compared to 100:60 in gel-SH hydrogel 1), was produced. The gel-SH had an average molecular weight of about 100 kDa (250 Bloom) and an average degree of substitution of about 0.8 mmol/g. The HA-MA (HA from Stanford Chemicals; functionalized by Blafar Ltd.) had an average molecular weight of about 33 kDa and an average degree of substitution of about 0.81 mmol/g. The gel-SH/HA-MA dry pre-mix was combined with a photoinitiator solution comprising eosin Y and triethanolamine in a saline solution. The resulting liquid biopolymer mixture comprised eosin Y at a concentration of 0.05 mM Eosin Y, triethanolamine at a concentration of about 0.076% w/v, gel-SH at a concentration of about 120 mg/ml and HA-MA at a concentration of about 50 mg/ml.

The procedure for preparing the liquid biopolymer mixture was as follows:
i. Weigh the dry (lyophilized) biopolymers (HA-MA and gel-SH) and mixing them in a vial in the desired weight ratio.
ii. Dissolve the lyophilized biopolymers in saline, with spinning (2000 g for 10 sec) within 5 min of adding saline and incubating in the dark at 37° C. until the biopolymers dissolve completely to form a clear solution (about 10-15 minutes).
iii. Add photoinitiator solution to the biopolymer solution.

The respective liquid biopolymer mixtures were converted into a crosslinked hydrogel by exposure to white light. Formula 1 was exposed to the light at an intensity of about 100 mW/cm² for about 6-8 minutes, and Formula 2 was exposed to the light at an intensity of about 10 mW/cm² for about 10 minutes.

For the hydrogel to serve as an HRC, it is desirable for the gel-SH hydrogel have physical properties that are similar to native corneal tissue. The above-noted formulated were shown to have physical properties that were similar to and compatible with native human cornea, as shown in the example below.

### Example 2—Testing of Hydrogels for Physical Characteristics

The hydrogels produced in accordance with Example 1 were then tested for various physical characteristics, including the following:

Transparency—The hydrogels were prepared in 96 well plate in triplicate and transmittance to visible light was assessed by recording the spectra in a plate reader (Enspire, Perkin Elmer). The obtained absorbance values were converted to transmittance by Beer Lambert's law, and plotted using saline as reference. Average transmittance was derived by averaging the values obtained for the complete spectra (400-700 nm).

Compressive modulus—Cylindrical hydrogels of 5 mm diameter and 1 mm height were prepared, and tested in a UTS instrument (BiSS mechanical tester) with parallel plate fixtures for compressive modulus assessment. The hydrogels were compressed at a rate of 1 mm/min up to a maximum of 50%, using a 44N load cell. The values for strain and load were recorded and the compressive modulus was calculated from the slope of stress versus strain curve, using linear region between 0.1 to 0.2 mm/mm strain.

Adhesive strength—The adhesion strength of the hydrogel to the biological tissue was assessed following the ASTM F2458-05 standards. The porcine skin tissues, purchased from a local butcher shop, were washed thoroughly to remove the oil and cut into 2.5 cm×1.5 cm pieces. The skin tissue pieces were glued to the glass slides. An incision was made between the two adjacent glass slides and the skin tissues were kept 1 mm apart and the gap was filled in by 75 μl pre-gel solution followed by photo-crosslinking. The assembly was incubated in saline for hydrogel equilibration followed by adhesion testing in the BiSS UTS instrument. The glass slides were fixed in the wedge grips (without disturbing the alignment). The assembly was stretched at a rate of 1 mm/min until adhesive or cohesive failure was observed. Adhesion strength was calculated from the relation: (Maximum load/cross-sectional area)×1000

Swelling by weight and volume—Hydrogel discs were prepared in the molds of specified geometry (5 mm diameter and 1 mm height) and incubated in normal saline for assessing the swelling via change in weight and volume with time. The weight measurements were performed by soaking the excess saline from the hydrogel and recording the weights at different time points. The swelling % by weight was measured using the relation: [(Wt−Wi)/Wi]×100. Where Wt is the weight recorded at different time points and Wi is the initial weight. Volumetric swelling was calculated by measuring the percentage increase in volume when the dimensions were measured using a Vernier caliper. The swelling % by volume was measured using the relation: [(Vt−Vi)/Vi]×100. Where Vt is the volume recorded at different time points and Vi is the initial volume.

Degradation—Degradation rate of the hydrogels was determined by incubating the hydrogels in 1× phosphate buffered saline (PBS) at ambient temperature under constant shaking (350 rpm) and measuring the change in weight with time. The degradation media was replenished every alternate day to avoid saturation and the hydrogel samples were retrieved at the predetermined time points and their weights were recorded after lyophilization for 24 h at −110° C. The degradation percentage was calculated using the relation: [(Wi−Wt)/Wt]×100. Where Wt is the dry weight at different time points and Wi is the initial dry weight recorded on the same day of sample preparation.

Burst pressure—Burst pressure of the hydrogels, which indicates, inter alia, the capability to withstand intraocular pressure, was measured using transplant rejected cadaveric cornea tissues. The tissues were cleaned and a 2 mm full thickness perforation was made at the center of the tissue. The perforation was filled with 8 μl pre-gel solution followed by photo-crosslinking. The hydrogel filled cornea was housed on the anterior chamber connected with a syringe pump. 1×PBS was flown to the assembly at a constant rate of 0.5 ml/min until the hydrogel unplugs from the site or starts leaking. Pressure was monitored using a wireless pressure sensor, and burst pressure was determined by calculating the difference between the initial and final pressure (maximum recorded value just before the unplugging).

Refractive index—Refractive index was measured using digital refractometer (Hanna Instruments), where the light passes through a prism in contact with the sample. An image sensor determined the critical angle at which the light refracts from the sample.

The resulting hydrogel demonstrated the following physical properties:

TABLE 1

| | Native cornea | | Formula 1 | Formula 2 |
|---|---|---|---|---|
| Gel-SH:HA-MA | n/a | | 100:60 | 120:50 |
| ratio (by weight) | | | | |
| Transparency | ~87% | | 86.06 ± | 91.18 ± |
| | | | 1.3% | 1.25% |
| Compressive | 100-300 | kPa | 352.41 ± | 346.49 ± |
| modulus | | | 25.82 kPa | 18.76 kPa |
| Adhesive strength | n/a | | 33.44 ± | 35.18 ± |
| | | | 1.22 kPa | 14.71 kPa |
| Maximum | n/a | | 9.38 ± 3.11% | 16.91 ± 4.17% |
| swelling: weight | | | (after 30 min) | (after 30 min) |
| Maximum | b/a | | 5.61 ± 2.2% | 9.15 ± 2.70% |
| swelling: volume | | | (after 30 min) | (after 30 min) |
| Degradation | b/a | | 7.2 ± 2.08% | 6.45 ± 1.53% |
| | | | (in 4 weeks) | (in 6 weeks) |
| Burst pressure | 2.5 | kPa | 27.57 ± | Not measured |
| | | | 8.2 kPa | |

| | | | |
|---|---|---|---|
| Refractive Index | 1.37 | 1.34 ± | 1.34 ± |
| | | 0.0001 | 0.001 |

The above physical properties demonstrate that the hydrogel produced in accordance with the above-described formulation performed equivalently or better than conventional biopolymers for mimicking properties of human cornea. In particular, the both gel-SH hydrogel formulations 1 and 2 showed lower swelling as measured by both weight and volume, had a compressive modulus more similar to native cornea, and showed less degradation in comparison with a conventional formulation. In addition, and unexpectedly, the refractive index of both gel-SH hydrogel formulations 1 and 2 was substantially the same as the refractive index of native cornea (1.37 in native cornea compared to 1.34 in both formulas),

In an aqueous solution with gel-SH, HA-MA and a photoinitiator, there is expected to be some spontaneous cross-linking, even in low light conditions. However, a low rate of spontaneous crosslinking may be advantageous such that the mixture maybe be kept in un-crosslinked form until the mixture is applied to the desired location, such as on the site of corneal injury. It was surprisingly found that Formula 2 advantageously had a slower rate of spontaneous cross-linking, such that the mixture was labile enough to be easily applied on a site of corneal injury for up to about 15 minutes after the mixture was prepared. By contrast, the Formula 1 was found to spontaneously crosslink at a faster rate such that that the mixture was labile enough to be easily applied on a site of corneal injury for up to about 5 minutes after the mixture was prepared.

### Example 3—Biocompatibility of Hydrogels for hCSSCs

For the gel-SH hydrogel to serve as an HRC and engender scarless tissue regeneration at the corneal wound site, it is important for the hydrogel to allow for corneal stromal stem cells to remain viable, as well as to maintain their phenotype and help in scar-less healing of the wound while gradually attaining the differentiated state. As such, biocompatibility of the hydrogel was tested by encapsulating human corneal stromal stem cells (hCSSCs) with the hydrogel and culturing the cells therein for four weeks. hCSSCs, after being passaged three times, were cultured either on a coverslip surface or within a hydrogel matrix. For coverslip culturing, the hCSSCs were mixed into a culture solution at a seeding density of 5000 cells/cm² and added to a well comprising a coverslip. For hydrogel culturing, the hCSSCs were mixed into a liquid biopolymer formulation at a seeding density of 3×10⁶ cells/ml, and the mixture was then crosslinked with light exposure to form a hydrogel. The crosslinked hydrogel thereby formed a culture medium for the hCSSCs encapsulated therein. The hydrogel was maintained at 37° C., 5% CO₂ sterile condition and monitored for 4 weeks. The cells on the coverslips or the cell-infused hydrogel were incubated in a culture medium recommended for CSSCs, and the culture medium was changed every third day.

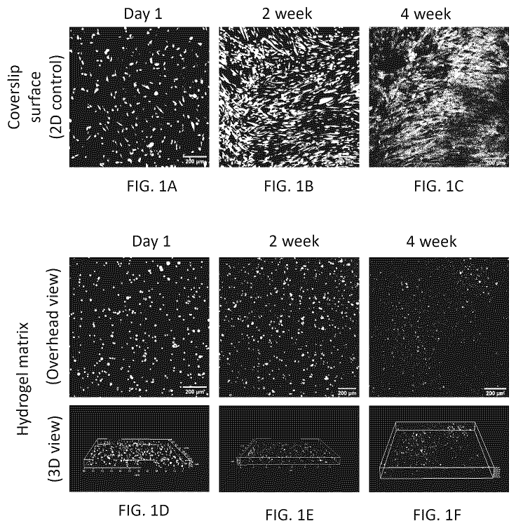

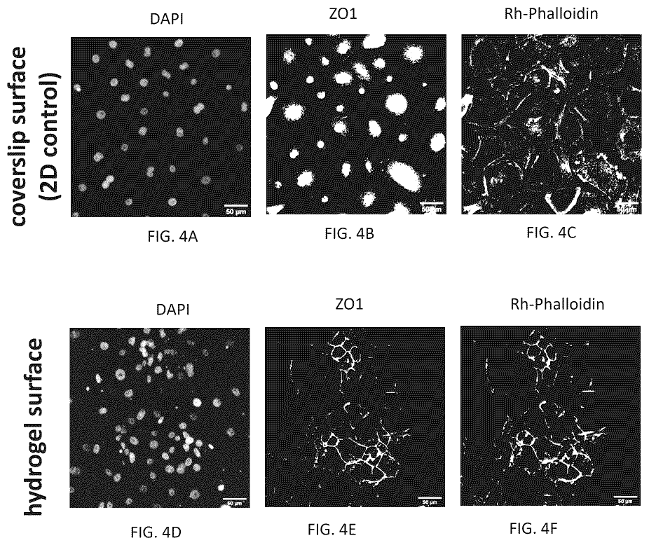

During the 4-week time, cell viability was assessed using Calcein-AM/Ethidium homodimer/DAPI staining. FIGS. 1A-1C FIGS. 1A-1C shows cells grown in the coverslip cultures at 1 day (FIG. 1A), 2 weeks (FIG. 1B) and 4 weeks (FIG. 1C) after culturing. FIGS. 1D-1F shows the overhead and 3D views of the cells grown in the hydrogel cultures based on Formula 2 of Example 1 at 1 day (FIG. 1D), 2 weeks (FIG. 1E) and 4 weeks (FIG. 1F) after culturing. As shown in FIGS. 1D-1F, cell distribution within the hydrogel was homogenous, and >80% of the cell population appeared viable after culturing for 4 weeks, thereby showing that the hydrogel was compatible with human corneal stromal cells. In addition, compared to the hCSSCs grown on coverslips,

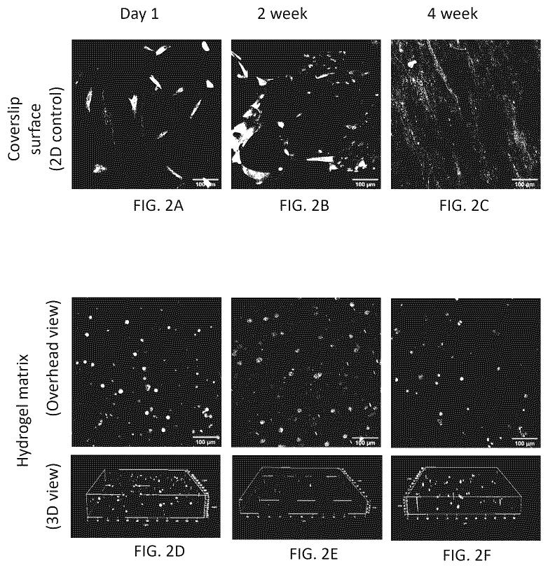

Cell phenotype was assessed using CD90/αSMA/DAPI staining. CD90 is a stromal stem cell biomarker, whereas the expression of αSMA by the cells would reflect their differentiated state to keratocytes or myofibroblasts. FIGS. 2A-2C shows an overhead view of the cells grown in the coverslip cultures at 1 day (FIG. 2A), 2 weeks (FIG. 2B) and 4 weeks (FIG. 2C) after culturing. FIGS. 2D-2F shows the overhead and 3D views of the cells grown in the hydrogel cultures based on Formula 2 of Example 1 at 1 day (FIG. 2D), 2 weeks (FIG. 2E) and 4 weeks (FIG. 2F) after culturing. The staining shows that the stromal stem cells initially maintained their stromal stem cell phenotype as indicated by CD90 staining, and gradually transitioned into a differentiated state as indicated by increased αSMA staining over 4 weeks.

### Example 4—Biocompatibility of Hydrogel for hCECs

For the gel-SH hydrogel to serve as an HRC and engender scarless tissue regeneration at the corneal wound site, it is also important for the hydrogel to allow growth for human corneal epithelial cells (hCECs). Gel-SH hydrogel (formula 2) was assessed for providing supporting surface for epithelial cell growth, by seeding primary hCECs on the hydrogel and allowing monolayer formation. hCECs suspension was seeded directly on the hydrogel surface or a coverslip control surface at the seeding density of 10 cells/cm². The culture was maintained at 37° C., 5% CO₂ sterile condition and monitored every day for a week. During the week's time, cell confluency was assessed using phase contrast imaging, and staining for the tight junction marker (Zona occludens—ZO1), along with DAPI and rhodamine-conjugated phalloidin.

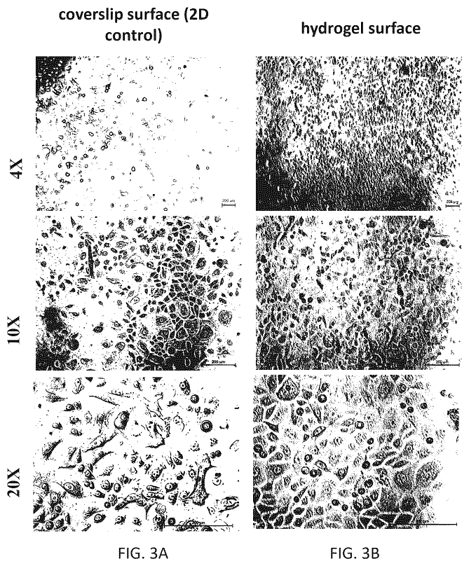

FIGS. 3A-3B show phase contrast images of the hCEC cells grown in the coverslip cultures after 4 days comparing hCECs grown on a coverslip surface (FIG. 3A) and on a hydrogel surface (FIG. 3B). The comparison shows that hCEC proliferation was more robust on the hydrogel surface, such that by 4 days, full confluency was reached on the hydrogel surface but not on the coverslip surface.

FIGS. 4A-4C show hCEC cells grown on a coverslip surface and stained with DAPI (FIG. 4A), ZO1 (FIG. 4B) and rhodamine-phalloidin (FIG. 4C). FIGS. 4D-4F show hCEC cells grown on a surface of a hydrogel based on Formula 2 of Example 1 and stained with DAPI (FIG. 4D), ZO1 (FIG. 4E) and rhodamine-phalloidin (FIG. 4F). The comparison of ZO1 staining shows that whereas growth on the hydrogel surface promoted tight junction formation in the hCECs by day 4 of culturing, growth on the coverslips did not.

1. A biopolymer formulation comprising:
a thiolated gelatin having an average molecular weight of between 50 kDa and 200 kDa, and an average degree of substitution of between about 0.2 mmol/g and about 1.2 mmol/g; and
a methacrylated hyaluronic acid having an average molecular weight of between 20 kDa and 80 kDa, and with an average degree of substitution of between about 0.5 mmol/g and about 1 mmol/g.

2-3. (canceled)

4. The biopolymer formulation of claim 1, wherein the thiolated gelatin is between about 100 bloom and about 300 bloom.

5. (canceled)

6. The biopolymer formulation of claim 1, wherein the methacrylated hyaluronic acid has an average molecular weight of between about 30 kDa and about 45 kDa.

7-8. (canceled)

9. The biopolymer formulation of claim 1, wherein the methacrylated hyaluronic acid and the thiolated gelatin are in a dr pre-mix preparation.

10. The biopolymer formulation of claim 1, wherein the biopolymer formulation comprises a saline solution and at least one photoinitiator compound, and wherein the thiolated gelatin, the methacrylated hyaluronic acid, and the at least one photoinitiator compound arm dissolved in the saline solution.

11. The biopolymer formulation of claim 10, wherein the biopolymer formulation is in the form of a liquid or a hydrogel.

12. (canceled)

13. The biopolymer formulation of claim 10, wherein the biopolymer formulation comprises the thiolated gelatin at a concentration of between about 50 mg/ml and about 140 mg/ml.

14. (canceled)

15. The biopolymer formulation of claim 10, wherein the biopolymer formulation comprises the methacrylated hyaluronic acid at a concentration of between about 40 mg/ml and about 90 mg/ml.

16. (canceled)

17. The biopolymer formulation of claim 1, wherein the thiolated gelatin and the methacrylated hyaluronic acid are at a weight ratio of between about 100:30 and about 100:70.

18. The biopolymer formulation of claim 1, wherein the biopolymer formulation comprises a therapeutic agent.

19. The biopolymer of claim 18, wherein the therapeutic agent is a therapeutic exosome.

20. A method of treating a disease of or damage to a cornea of a subject, the method comprising:
providing a liquid biopolymer mixture comprising at least one photoinitiator compound;
applying the liquid biopolymer mixture to the cornea of a subject; and
exposing the liquid biopolymer mixture applied on the cornea to light having a wavelength within an excitation range of the at least one photoinitiator compound,
wherein the liquid biopolymer mixture comprises:
a thiolated gelatin having an average molecular weight of between 50 kDa and 200 kDa, and an average degree of substitution of between 0.2 mmol/g and 1.2 mmol/g; and
a methacrylated hyaluronic acid having an average molecular weight of between 20 kDa and 80 kDa, and with an average degree of substitution of between 0.5 mmol/g and 1 mmol/g.

21. The method of claim 20, wherein the at least one photoinitiator compound comprises eosin Y and triethanolamine, and the light is white light.

22. The method of claim 20, wherein the liquid biopolymer mixture is produced by mixing a dry biopolymer pre-mix comprising the thiolated gelatin and the methacrylated hyaluronic acid with a photoinitiator solution comprising the at least one photoinitiator compound dissolved into a saline solution.

23. The method of claim 20, wherein the liquid biopolymer mixture comprises a therapeutic agent.

24. The method of claim 23, wherein the therapeutic agent is a therapeutic exosome.

25. A biopolymer formulation comprising:
a thiolated gelatin having a bloom value of between about 200 bloom and about 275 bloom; and
a methacrylated hyaluronic acid having an average molecular weight of between 25 kDa and 40 kDa, and an average degree of substitution of between about 0.7 mmol/g and about 0.9 mmol/g,
wherein the thiolated gelatin and the methacrylated hyaluronic acid are at a weight ratio of between about 100:30 and about 100:70.

26-33. (canceled)

34. A liquid biopolymer mixture comprising:
an aqueous solvent;
at least one photoinitiator compound;
a thiolated gelatin having an average molecular weight of between 50 kDa and 200 kDa; and
a methacrylated hyaluronic acid having an average molecular weight of between 20 kDa and 80 kDa, and with an average degree of substitution of between about 0.5 mmol/g and about 1 mmol/g,
wherein the mixture is in the form of a liquid or a hydrogel.

35. The liquid biopolymer mixture of claim 34, wherein the biopolymer formulation comprises the thiolated gelatin at a concentration of between about 50 mg/ml and about 140 mg/ml, and the methacrylated hyaluronic acid at a concentration of between about 40 mg/ml and about 90 mg/ml.

36. The biopolymer formulation of claim 34, wherein the liquid biopolymer mixture comprises a therapeutic exosome.
